(19) United States
(12) Patent Application Publication
(10) Pub. No.: US 2022/0354515 A1
(43) Pub. Date: Nov. 10, 2022

(54) ENDOSCOPIC SIDE SNARE TOOLS AND METHODS FOR USE

(71) Applicant: EndoscopyTools, LLC., Carmel, IN (US)

(72) Inventor: Mazen Alsatie, Carmel, IN (US)

(21) Appl. No.: 17/739,336

(22) Filed: May 9, 2022

Related U.S. Application Data
(60) Provisional application No. 63/186,049, filed on May 7, 2021.

(51) Int. Cl. A61B 17/221; A61B 17/00

(52) U.S. Cl. CPC A61B 17/221; A61B 17/00234; A61B 2017/2212; A61B 2017/00358

(57) ABSTRACT

Endoscopic tools or instruments having one or more side-deployable snare loop tools are provided. Loop wire tools connected by cables to proximal controlling handles are introduced though side openings in the tools which are located proximal to the distal end of the tools which may also include a soft, blunt tip designed to rest against a body cavity wall for stability. Side introduction of the wire loop tools into a human cavity allows for angulation and positioning favorable to perform endoscopic intervention, particularly when dealing with elongated foreign bodies or impacted food in the esophagus.

### CROSS REFERENCE TO RELATED APPLICATIONS

This application is related to U.S. Provisional Patent Application No. 63/186,049 filed on May 7, 2021, titled “Endoscopic Side Snare Tools and Methods for Use” which is incorporated herein by reference.

### FIELD

The claimed technology relates generally to medical instruments and more particularly towards medical instruments for use in endoscopic procedures.

### BACKGROUND

An endoscope is an instrument that can be inserted into a body cavity to provide a visual account of the cavity proximate the instrument's distal or remote end. The instrument can also be equipped to carry out certain surgical procedures and the like at its distal end utilizing tools inserted through the instrument's tubular channel. The typical tubular instrument is relatively flexible to allow its tip to be maneuvered to a site of interest. The tip of the instrument is equipped with both a light source and optics in communication with the instrument's proximal end. The visual information obtained at the distal end or tip of the instrument is carried to the other end of the instrument where it can be viewed through a computer screen. Other controls at the proximal end of the instrument control the instrument's tip through cables or filaments running through the endoscope's tubular structure.

Endoscopes currently available can be equipped to provide a visual inspection of bodily cavities, and to carry out certain surgical operations and other minor medical procedures. The distal or remote and flexible end of the instrument can be moved to a limited extent by turning knobs or pulling cables which bend the tube in a desired direction.

Food impaction in the esophagus is a common gastrointestinal emergency. There are several tools for removal of impacted food. The procedure for food removal can be tedious and several tools are utilized mainly due to the size of the food bolus impacted in the esophagus. Cutting the food bolus into smaller pieces has proven to be a generally safe method to relief the impaction. With current tools, this process frequently laborious and regular snare is not generally successful in achieving this goal.

Foreign body in the gastrointestinal tract is a common gastrointestinal emergency. There are several tools for removal. Elongated foreign body such as a pencil or toothbrush in a body cavity can be difficult to remove and may lead to complications if both ends were not controlled well during removal. The current disclosure addresses the shortcomings of current endoscopic instruments.

### SUMMARY

In one aspect an endoscopic tool for use with an endoscope device positioned within the body cavity of a patient. The endoscopic tool having a proximal portion and a distal portion connected by a middle portion and covered in a flexible sheath with a longitudinal axis running therethrough. The proximal end has a control member operationally connected thereto. The middle portion having a generally tubular channel running therethrough. The distal portion having a blunt tip and an opening transverse to the longitudinal axis and operationally connected to the channel of the middle portion. Further included is a wire loop tool disposed in the channel and having a proximal end, a control cable, and a loop end. The proximal end of the wire loop tool being operationally connected to the control member of the proximal end such that extending the wire loop tool extends the loop end through the distal portion opening transverse to the longitudinal axis.

In another aspect the endoscopic tool further includes a second control member disposed on the proximal end, a second channel disposed in the middle portion, and a second opening in the distal portion transverse to the longitudinal axis and operationally connected to the second channel. Further, a second wire loop tool is disposed in the second channel and has a proximal end, a control cable, and a loop end. The proximal end of the second wire loop tool being operationally connected to the second control member such that extending the second loop wire tool extends the second loop end through the distal portion second opening transverse to the longitudinal axis.

In still a further aspect an endoscopic tool for use with an endoscope device positioned within a body cavity of a patient is provided. The endoscopic tool having an elongated generally tubular tool body with a proximal and distal end and a longitudinal axis running therethrough. A control member is disposed at a proximal end of the tool for controlling the tool. A wire loop tool having a proximal end, a control cable portion, and a wire loop end is also provide where the proximal end is operationally connected to the control member. A tubular channel is disposed along the longitudinal axis of the tool where the wire loop tool is disposed within the channel such that the wire loop end is within the channel when in a fully retracted position and extends from the channel when in an extended position. The endoscopic tool further includes a blunt tip portion coupled to the distal end of the tool body. An opening is disposed in the side of the tubular channel and arranged such that the wire loop extends from the opening transverse to the longitudinal axis of the tool when in the extended position.

In yet another aspect a second control member is disposed on the proximal end and a second wire loop tool having a proximal end, a control cable portion, and a wire loop end, the proximal end is operationally connected to the second control member. A second tubular channel is disposed along the longitudinal axis of the tool, the second wire loop tool being disposed within the second channel such that the second wire loop end is within the second channel when in a fully retracted position and extends from the second channel when in an extended position. A second opening being disposed in the side of the second tubular channel and arranged such that the second wire loop extends from the second opening transverse to the longitudinal axis of the tool when in the extended position.

### BRIEF DESCRIPTION OF THE DRAWINGS

FIG. 1 is a side diagrammatic view of an endoscope according to the prior art.

FIG. 2 is front diagrammatic view of an endoscope tip according to the prior art

FIG. 3 is side partial cross sectional diagrammatic view of an endoscopic tool according to one example of the disclosed invention.

FIG. 4 is side partial cross sectional diagrammatic view of an endoscopic tool according to another example of the disclosed invention.

FIG. 5 is side partial cross sectional diagrammatic view of an endoscopic tool according to still another example of the disclosed invention.

### DESCRIPTION

For the purposes of promoting an understanding of the principles of the claimed technology and presenting its currently understood best mode of operation, reference will now be made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates.

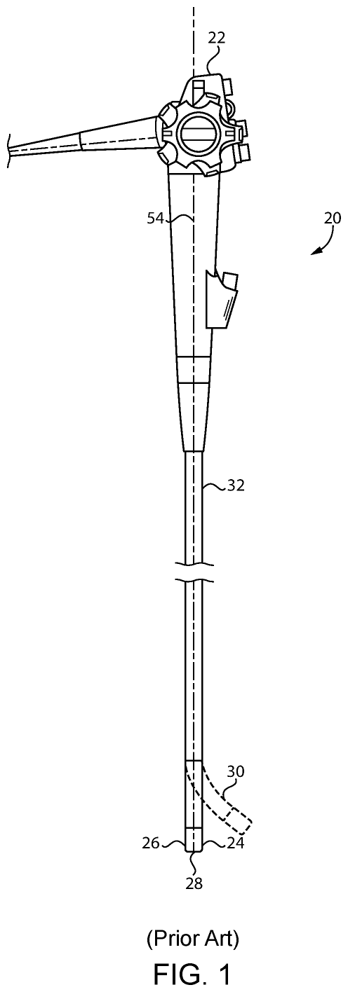

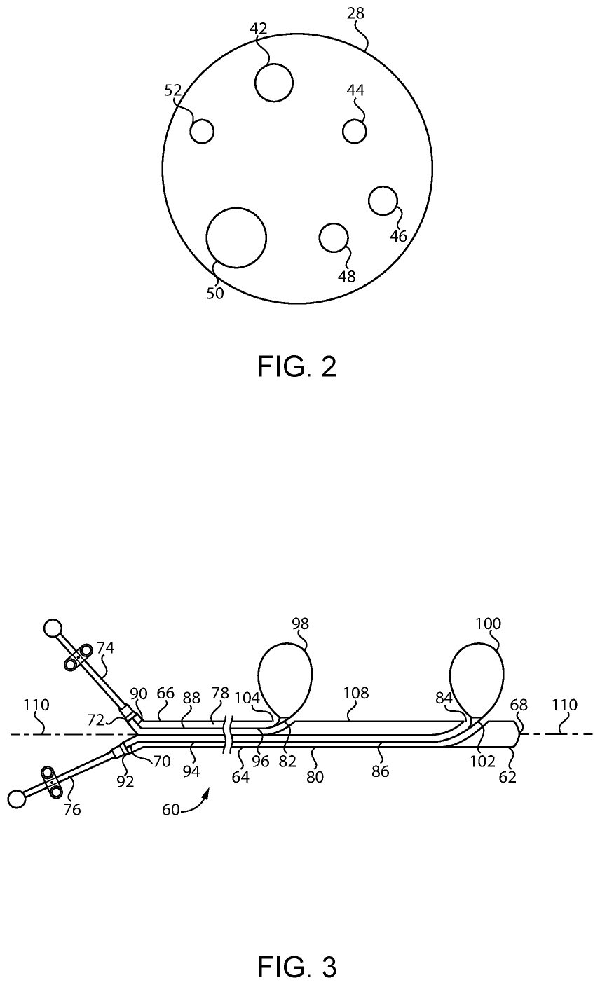

An example of an endoscopic instrument 20 according to the prior art is shown in FIGS. 1-2. The endoscopic instrument 20 has a proximal end 22 and a distal end 26 connected by a middle portion 32. The distal end 26 having a soft tip portion 24 and an end face 28 (shown in greater detail in FIG. 2). The distal portion 26 is generally flexible allowing the distal end to flex 30 away from a center line 54 of the instrument 20. The instrument body includes a plurality of tubes or channels which travel from the proximal end 22, through the middle portion 32, and terminate at the end face 28 of the distal end 26. These channels are designed to allow passage of instruments and/or materials therethrough to assist the user in performing a procedure using the instrument 20. Examples include a light 52 which provides illumination within the body cavity which allows a user to observe the inside of the cavity via a lens 42. One or more of the channels may be configured to provide water and/or air into the body cavity using a water nozzle 46 or an air nozzle 48. One or more instrument channels 50 pay also be provided to allow a user to introduce and manipulate instruments within the body cavity. The type and nature of instruments may vary according to the particular procedure, but may include cutting instruments, traction wires/clips, wire loops, balloons, and the like. The exact number and/or configuration of channels and openings in the end face may differ from those shown in FIG. 2.

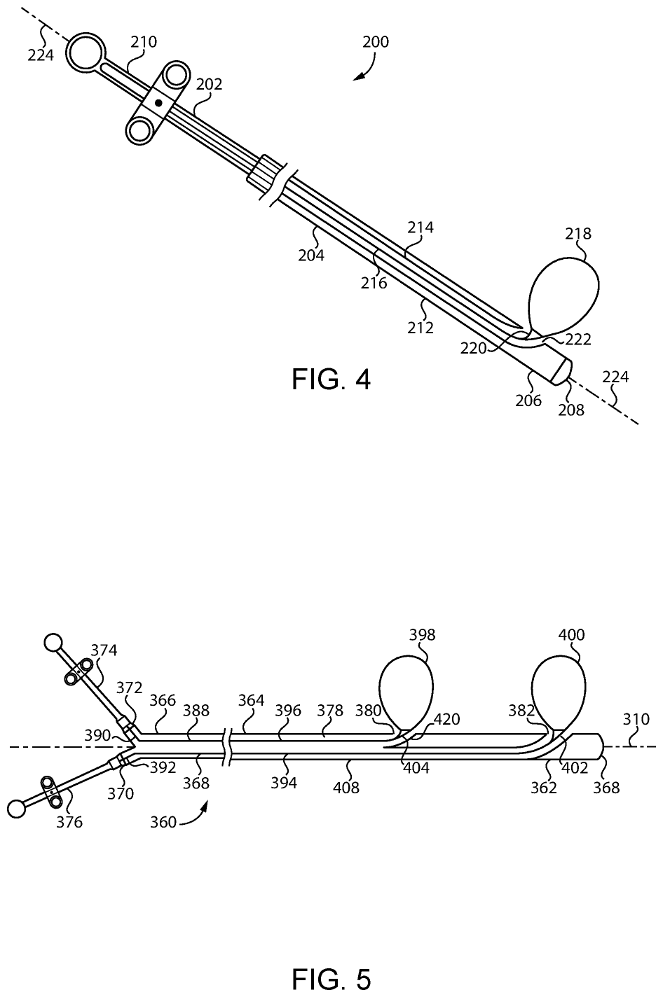

This current disclosure describes a new design of endoscopic tools. In this new design, the functional mobile components of the tool are positioned on the side of the tip, thus altering the axis of projection of the tool's components. Embodiments of this endoscopic tool substantially facilitates interventions inside the gastrointestinal tract. This current disclosure provides a unique design of an endoscopic tool having a single (FIG. 4) or double (FIGS. 3, 5) side snare positioned at the side of the device's distal end. The positioning of the side snare facilitates the process of reaching, grasping each end of an object independently, thereby effecting removal of an elongated foreign body from a body cavity. Furthermore, this positioning of the snare(s) facilitates the process of reaching, cutting, and removing impacted food in the esophagus. Other applications of the disclosed tools may also be used in other procedures.

FIG. 3 shows a partial cross sectional diagrammatic view of an endoscopic tool 60 according to one example of the disclosed invention. In this particular example, the endoscopic tool 60 has a distal portion 62 and a proximal portion 66 joined by a middle portion 64 which run along a longitudinal axis 110. The body of the tool 60 comprises a flexible sheath 108 which allows the tool 60 to be flexed and/or displaced off its longitudinal axis 110. The distal portion 62 includes a blunt tip 68. The proximal portion 66 includes two control members 74, 76 connected to proximal channel openings 70, 72. In some examples, the control members are removably connected to the proximal channel openings. The middle portion 64 includes a first channel 78 and a second channel 80 which are generally tubular. The first channel 78 begins at a first proximal channel opening 72 and terminates at first channel distal opening 82 in the sheath 108. The second channel 80 begins at a second proximal channel opening 70 and terminates at second channel distal opening 84 in the sheath 108. The first and second distal channel openings are arranged so as to be transverse to the axis 110 of the tool 60. That is, first and second distal channel openings are arranged such that a wire exiting the openings would be generally orthogonal to the axis 110 of the tool 60. The exact distance between the first channel distal opening 82 and the second channel distal opening 84 may vary from example to example. Typically, the first channel distal opening 82 and the second channel distal opening 84 will be from 95-145 mm apart. The exact distance between the first channel distal opening 82 and blunt tip 68 may vary from example to example, but is typically 100-150 mm. The exact distance between the second channel distal opening 84 and blunt tip 68 may vary from example to example, but is typically 3-5 mm. These distances may be greater or lesser as desired. For example, the openings may be closer together for specific applications. The overall length of the instrument will typically be 1800-2300 mm, but may be longer or shorter as desired. For example, endoscopic tools such as those disclosed herein for use with pediatric patients may be shorter overall.

Disposed within each of the first and second channels 78, 80 are wire loop tools 86, 88. Each of the wire loop tools 86, 88 includes a proximal end 90, 92 which are operationally connected to a control member cable 96, 94, a control member cable 96, 94 which are operationally connected to a distal wire loop portion 100, 98 by a flexible joint 102, 104. The proximal end 90 of the first wire loop tool 88 is operationally connected to the first control member 74. The proximal end 92 of the second wire loop tool 86 is operationally connected to the second control member 76. Operation of a control member allows a user to open, close, and otherwise manipulate the associated distal wire loop through the distal channel openings transverse to the central axis of the tool. Such manipulations may include extending the wire loop from a channel through the channel opening and into a body cavity, opening/closing the wire loop, twisting or otherwise providing angulation of the wire loop relative to the longitudinal axis of the tool, collapsing the loop and withdrawing it into a channel of the tool, and the like.

FIG. 4 shows a partial cross sectional diagrammatic view of an endoscopic tool 200 according to another example of the disclosed invention. In this particular example, the endoscopic tool 200 has a proximal portion 202 and a distal portion 206 connected by a middle portion 204 which are aligned along a longitudinal axis 224. The body of the tool 200 comprises a flexible sheath 212 which allows the tool 200 to be flexed and/or displaced off its longitudinal axis 224. The distal portion 206 includes a blunt tip 208 which may be rounded and/or include a soft covering or coating (such as silicone). The proximal portion 202 includes a control member 210 which is connected to a proximal channel opening (not shown) similar to the proximal channel openings described and shown in FIG. 3. The middle portion 204 includes a channel 214 which is in communication with the proximal channel opening and a distal channel opening 222. Disposed within the channel 214 is a wire loop tool 216 having a proximal end (not shown) operationally connected to the control member 210 and a distal end operationally connected to a wire loop portion 218 at a flexible joint 220. Operation of the control member 210 allows a user to open, close, and otherwise manipulate the distal wire loop 218 through the distal channel opening 222 transverse to the central axis 224 of the tool 200. Such manipulations may include extending the wire loop from the channel through the channel opening and into a body cavity, opening/closing the wire loop, twisting or otherwise providing angulation of the wire loop relative to the longitudinal axis of the tool, collapsing the loop and withdrawing it into the channel of the tool, and the like.

Operation of tools according to the present disclosure such as those shown in FIGS. 3-4 will now be described. The following are examples of methods of using the disclosed endoscopic tools and it is understood that other methods of using the disclosed endoscopic tools may also be employed depending on a variety of factors such as the procedure being performed, the specific design and operation of the endoscope being used, and the like.

FIG. 5 shows a partial cross sectional diagrammatic view of an endoscopic tool 360 according to still another example of the disclosed invention. This particular example is similar to that shown in FIG. 3 in that it includes two wire loop tools, however, it uses only a single channel in the body of the tool to house both wires. The endoscopic tool 360 has a distal portion 362 and a proximal portion 366 joined by a middle portion 364 which run along a longitudinal axis 310. The body of the tool 360 comprises a flexible sheath 408 which allows the tool 360 to be flexed and/or displaced off its longitudinal axis 410. The distal portion 362 includes a blunt tip 368. The proximal portion 366 includes two control members 374, 376 connected to proximal channel openings 370, 372. In some examples, the control members are removably connected to the proximal channel openings. The middle portion 364 includes a tool channel 378 which is generally tubular. The tool channel 378 begins at the proximal channel openings 370, 372, includes a first distal opening 380, and terminates at the second distal opening 382 in the sheath 408. The first and second distal tool channel openings are arranged so as to be transverse to the axis 110 of the tool 360. That is, first and second distal tool channel openings are arranged such that a wire exiting the openings would be generally orthogonal to the axis 410 of the tool 360. The exact distance between the first distal opening 380 and the second distal opening 382 as well as the distance between the second distal opening 382 and the blunt tip 368 may vary from example to example.

Disposed within tool channel 378 is wire loop tools 386, 388. Each of the wire loop tools 386, 388 includes a proximal end 390, 392 which are operationally connected to a control member cable 396, 394, a control member cable 396, 394 which are operationally connected to a distal wire loop portion 400, 398 by a flexible joint 402, 404. The proximal end 390 of the first wire loop tool 388 is operationally connected to the first control member 374. The proximal end 392 of the second wire loop tool 386 is operationally connected to the second control member 376. A tool wire guide 420 is disposed within the tool channel 378 to direct the wire loop tool 388 through the first distal opening 380 and to prevent one wire from interfering in the other wire's operation and manipulation. The exact size and configuration of the tool wire guide 420 may vary. Optionally, the tool channel 378 may include further supports or dividers to keep the wire tools separated. Operation of a control member allows a user to open, close, and otherwise manipulate the associated distal wire loop through the distal channel openings transverse to the central axis of the tool. Such manipulations may include extending the wire loop from the tool channel through the channel opening and into a body cavity, opening/closing the wire loop, twisting or otherwise providing angulation of the wire loop relative to the longitudinal axis of the tool, collapsing the loop and withdrawing it into a channel of the tool, and the like.

In one example, an endoscopic tool such as those disclosed herein is inserted into a tool channel of an endoscope such as that shown in FIGS. 1-2. The wire loop tools are in the fully retracted position at this point and fully within the channels. Once the endoscope is positioned the tool is introduced into the body cavity by extending it through the tool lumen of the endoscope. The blunt tip of the tool may then be optionally positioned so as to rest against a portion of the cavity wall (e.g., the stomach wall if the procedure is to remove an object that had been swallowed) to provide stability to the tool while the wire tools are deployed and operated. Optionally, the blunt tip may include a soft covering or coating to prevent the tip from accidentally damaging the body cavity wall it rests against. The tool is then positioned as desired such that both the first and second distal openings are beyond the body of the endoscope so that the wire loop tools may be deployed. Each wire loop tool is then deployed such that the wire loop is partially or entirely outside of its respective channel and manipulated individually using the associated control member. For example, in a procedure to remove a swallowed object one of the wire loop tools may be deployed to initially capture the object then the second wire loop tool deployed and manipulated to secure the object against the body of the endoscopic tool. Such an arrangement of having two points of contact and fixture allows for the object to be better manipulated into a desired orientation prior to removal of the object and the tool. Having the wire loop tools deploy from the side of the tool rather than from the end also allows for better positioning of the object being removed. That is, the object may be held against the side of the tool body in a generally parallel orientation whereas with an end-deployed wire tool the object might drift into a more perpendicular orientation relative to the tool and endoscope bodies thereby making extraction more difficult. Optionally, prior to removal of the object and the tool the object may be cut into two or more smaller pieces to facilitate in removal. In such examples, capture by the two wire loops of the tool facilitates stabilizing the object while cutting in addition to effecting removal of the pieces.

While the claimed technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the claimed technology are desired to be protected.

What is claimed is:

1. An endoscopic tool for use with an endoscope, comprising:
a proximal portion and a distal portion connected by a middle portion and covered in a flexible sheath and having a longitudinal axis running therethrough;
the proximal end having a control member;
the middle portion connected to the proximal end and having a channel therethrough;
the distal portion having a blunt tip and an opening transverse to the longitudinal axis and operationally connected to the channel; and
a wire loop tool disposed in the channel and having a proximal end, a control cable, and a loop end, the proximal end operationally connected to the control member such that extending the wire loop tool extends the loop end through the distal portion opening transverse to the longitudinal axis.

2. The tool of claim 1, wherein the blunt tip includes a coating whereby biological tissue within a body cavity contacted by the tip is not damaged.

3. The tool of claim 2 wherein the coating is silicone.

4. The tool of claim 1 wherein the distal portion opening is 3-5 mm from the blunt tip.

5. The tool of claim 1 wherein the middle portion and distal portion are sized to pass through an instrument channel of an endoscope.

6. The tool of claim 1, further comprising:
a second control member disposed on the proximal end;
a second channel disposed in the middle portion;
a second opening in the distal portion transverse to the longitudinal axis and operationally connected to the second channel;
a second wire loop tool disposed in the second channel and having a proximal end, a control cable, and a loop end, the proximal end operationally connected to the second control member such that extending the loop wire tool extends the loop end through the distal portion second opening transverse to the longitudinal axis.

7. The tool of claim 6, wherein the distal portion second opening is 100-150 mm from the blunt tip.

8. An endoscopic tool for use with an endoscope device, comprising:
an elongated generally tubular tool body having a proximal and distal end and a longitudinal axis running therethrough;
a control member disposed at a proximal end of the tool for controlling the tool;
a wire loop tool having a proximal end, a control cable portion, and a wire loop end, the proximal end being operationally connected to the control member;
a tubular channel disposed along the longitudinal axis of the tool, the wire loop tool disposed within the channel such that the wire loop end is within the channel when in a fully retracted position and extends from the channel when in an extended position;
a blunt tip portion coupled to the distal end of the tool body; and
an opening disposed in the side of the tubular channel and arranged such that the wire loop extends from the opening transverse to the longitudinal axis of the tool when in the extended position.

9. The tool of claim 8, wherein the blunt tip includes a coating whereby biological tissue within a body cavity contacted by the tip is not damaged.

10. The tool of claim 8 wherein the tubular channel opening is 3-5 mm from the blunt tip.

11. The tool of claim 8 wherein tool body is sized to pass through an instrument channel of an endoscope.

12. The tool of claim 8, further comprising:
a second control member disposed on the proximal end;
a second wire loop tool having a proximal end, a control cable portion, and a wire loop end, the proximal end being operationally connected to the second control member;
a second tubular channel disposed along the longitudinal axis of the tool, the second wire loop tool disposed within the second channel such that the second wire loop end is within the second channel when in a fully retracted position and extends from the second channel when in an extended position; and
a second opening disposed in the side of the second tubular channel and arranged such that the second wire loop extends from the second opening transverse to the longitudinal axis of the tool when in the extended position.

13. The tool of claim 12, wherein the second opening is 100-150 mm from the blunt tip.

14. A method for using the endoscopic tool of claim 8, comprising:
inserting an endoscopic tool through the instrument channel of an endoscope the end of which has been positioned within the body cavity of a patient;
extending the endoscopic tool into the body cavity of a patient;
manipulating the control member to extend the wire loop through the opening in the side of the tubular channel;
manipulating the control member to capture an object within the body cavity with the wire loop;
withdrawing the endoscopic tool to remove the object from the body cavity.

15. The method of claim 14, wherein extending the endoscopic tool into the body cavity of a patient further includes contacting the blunt tip with a wall of the body cavity.

16. The method of claim 14, further comprising cutting the object into smaller pieces prior to withdrawing the endoscopic tool to remove the object from the body cavity.
